(19) United States
(12) Patent Application Publication
(10) Pub. No.: US 2023/0233142 A1
(43) Pub. Date: Jul. 27, 2023

(54) METHOD FOR SLEEP MONITORING, ELECTRONIC DEVICE AND NON-TRANSITORY COMPUTER-READABLE MEDIUM

(71) Applicant: GUANGDONG OPPO MOBILE TELECOMMUNICATIONS CORP., LTD., Dongguan (CN)

(72) Inventor: Haizhou YAN, Dongguan (CN)

(21) Appl. No.: 18/192,103

(22) Filed: Mar. 29, 2023

Related U.S. Application Data
(63) Continuation of application No. PCT/CN2021/107404, filed on Jul. 20, 2021.

(30) Foreign Application Priority Data
Sep. 29, 2020 (CN) 202011052065.2

(51) Int. Cl. A61B 5/00; A61B 5/024; A61B 5/11

(52) U.S. Cl. CPC A61B 5/4809; A61B 5/6898; A61B 5/02405; A61B 5/11

(57) ABSTRACT

Disclosed are a method and apparatus for sleep monitoring, an electronic device and a computer readable medium, relating to the field of health monitoring technology, the method comprises: a screen state of a target terminal is obtained; physiological parameters of a user are obtained, the physiological parameters including at least one of body motion parameters and heart rate parameters; a sleep state of the user is determined based on the screen state and the physiological parameters. Since the screen state can reflect the user's operation of the user terminal, and the operation can further reflect the sleep state of the user, and the physiological parameters can also reflect the sleep state of the user, the combination of the screen state and the physiological parameters can make the determination of sleep state more accurate.

### CROSS-REFERENCE TO RELATED APPLICATIONS

This application is a continuation of International Patent Application No. PCT/CN2021/107404, filed Jul. 20, 2021, which claims priority to Chinese Patent Application No. 202011052065.2, filed Sep. 29, 2020, the entire disclosures of which are incorporated herein by reference.

### TECHNICAL FIELD

The present application relates to the field of health monitoring technologies, and more specifically, to a method and apparatus for sleep monitoring, an electronic device, and a computer-readable medium.

### RELATED ART

With the popularity of wearable devices or mobile terminals such as smart phones, smart watches, smart bracelets (abbreviated as: “bracelets”), sleep health monitoring technology has gradually matured. However, the current accuracy of sleep monitoring smart devices is not particularly high, especially for users that play with cell phones in bed.

### SUMMARY

A method for sleep monitoring, an electronic device and a non-transitory computer-readable medium are provided in the embodiments of the present disclosure.

In a first aspect, an embodiment of the present application provides a method for sleep monitoring. The method can be performed by an electronic device, and include: a screen state of a target terminal is acquired; physiological parameters of a user are acquied, the physiological parameters including at least one of body motion parameters and heart rate parameters; a sleep state of the user is determined based on the screen state and the physiological parameters, the sleep state including a sleeping state and an awake state.

In a second aspect, an embodiment of the present application further provides an electronic device for sleep monitoring including one or more processors and a memory. The memory can be configured to store computer program code of one or more applications, which when executed by the processors, causes the processors to: acquire the screen state of a target terminal; acquire physiological parameters of a user, the physiological parameters including at least one of body motion parameters and heart rate parameters; determine a sleep state of the user based on the screen state and the physiological parameters, the sleep state including a sleeping state and an awake state.

In a third aspect, an embodiment of the present application further provides a non-transitory computer-readable medium, the non-transitory computer-readable medium storing program code executable by a processor, which when executed by the processor, causes the processor to: acquire a screen state of a target terminal; obtain physiological parameters of a user, the physiological parameters including at least one of body motion parameters and heart rate parameters; and determine a sleep state of the user based on the screen state and the physiological parameters, the sleep state comprising a sleeping state and an awake state.

The method for sleep monitoring, the electronic device and the non-transitory computer-readable medium described in the present application can obtain the screen state of the target terminal and the physiological parameters of the user, then, determine the sleep state of the user based on the screen state and the physiological parameters. The physiological parameters can be at least one of body motion parameters and heart rate parameters.

### BRIEF DESCRIPTION OF DRAWINGS

FIG. 1 illustrates a curve of variation of acceleration values in a sleep state provided by an embodiment of the present application.

FIG. 2 illustrates a curve of variation of heart rate in a sleep state provided by an embodiment of the present application.

FIG. 3 illustrates a method flowchart of a method for sleep monitoring provided by an embodiment of the present application.

FIG. 4 illustrates a method flowchart of a method for sleep monitoring provided by another embodiment of the present application.

FIG. 5 illustrates a diagram of a sleep period determined based on acceleration values provided by an embodiment of the present application.

FIG. 6 illustrates a diagram of a sleep period determined based on heart rate provided by an embodiment of the present application.

FIG. 7 illustrates a diagram of a sleep period determined based on screen state provided by an embodiment of the present application.

FIG. 8 illustrates a method flowchart of a method for sleep monitoring provided by another embodiment of the present application.

FIG. 9 illustrates a diagram of a sleep period determined based on HRV provided by an embodiment of the present application.

FIG. 10 illustrates a modular block diagram of an apparatus for sleep monitoring provided by an embodiment of the present application.

FIG. 11 illustrates a modular block diagram of an electronic device provided by an embodiment of the present application.

FIG. 12 illustrates a storage unit provided by an embodiment of the present application configured to save or carry program code implementing an information processing method of an embodiment of the present application.

### DETAILED DESCRIPTION

The following detailed description refers to the accompanying drawings. The same reference numbers may be used in different drawings to identify the same or similar elements. In the following description, for purposes of explanation and not limitation, specific details are set forth such as particular structures, architectures, interfaces, techniques, etc. in order to provide a thorough understanding of the various aspects of various embodiments. However, it will be apparent to those skilled in the art having the benefit of the present disclosure that the various aspects of the various embodiments may be practiced in other examples that depart from these specific details. In certain instances, descriptions of well-known devices, circuits, and processes are omitted so as not to obscure the description of the various embodiments with unnecessary detail.

With the accelerated pace of life and increased work pressure, sleep disorders have begun to become a major problem for modern people, with almost more than 60% of people having sleep disorders. There are numerous types of sleep disorders that can be broadly categorized into three main categories: difficulties falling asleep or staying asleep (insomnia), poor quality sleep, and sleep deprivation. These disorders can significantly disrupt our daily lives, and may lead to accelerated aging, weakened immune system, and increased risk of cardiovascular disease. Therefore, it is important to maintain healthy sleep habits.

The concept of preventive medicine is currently used to address sleep disorders, i.e., early detection and early treatment. The traditional method of assessing sleep disorders is to fill out a sleep assessment form or to do a full night of PSG monitoring. Although these two methods are quite accurate, it is not practical to do long-term monitoring considering their cost and inconvenience. Therefore, there is an urgent need for a convenient and inexpensive sleep monitoring system to help identify sleep disorders.

Due to the rapid development of wearable devices and smartphones, more and more sleep monitoring systems have been developed, which can be broadly classified into the following categories: acceleration signal-based, heartbeat signal-based, and joint detection of acceleration and heartbeat signals.

There are many shortcomings with current methods for sleep monitoring. For example, while the methods mentioned above can accurately monitor a user's sleep state in some situations such as when the user is lying in bed to sleep, they may not be as accurate in special situations where the user is lying in bed but remains awake while engaging in activities such as playing on their phone or watching videos. In these situations, the user's movements may be minimal and the monitoring results obtained using acceleration signals, heartbeat signals or a combination of both may mistakenly indicate that the user is asleep.

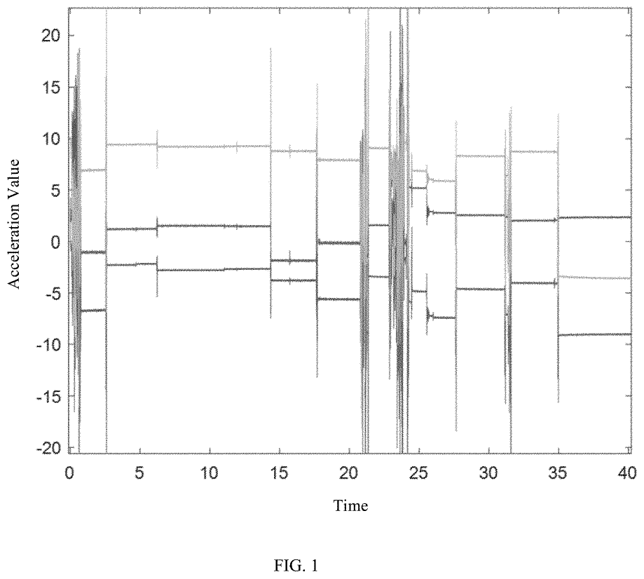

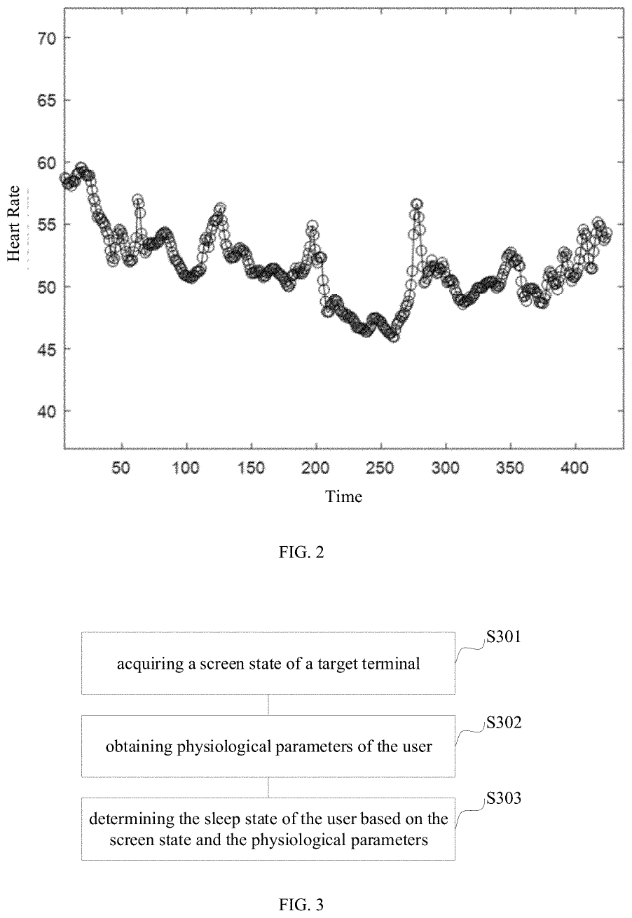

For example, a user is lying in bed watching a video or reading an article on a cell phone, an acceleration signal of the user's body, which is obtained by detection, is illustrated in FIG. 1, and a heart rate result which is detected is illustrated in FIG. 2. It can be seen that the acceleration value in FIG. 1 shows that the user has very little body movement at this time, and the body is almost in a stationary state. As can be seen from the heart rate values in FIG. 2, the heart rate of the user is quite low and smooth at this time, and the physical characteristics of the user while sleeping are that the body is quite stationary, with less body movement and a lower heart rate, i.e., stable at a resting heart rate. Therefore, it can be seen from FIG. 1 and FIG. 2 that the sleep monitoring results obtained by acceleration and/or heart rate at this time are asleep, however, the state of the user at this time is lying in bed and watching videos on a cell phone. Therefore, the sleep monitoring results do not match with the actual sleep state of the user, i.e., the monitoring result is wrong.

Therefore, in order to improve the above defects, as illustrated in FIG. 3, an embodiment of the present application provides a method for sleep monitoring, which may be performed by a mobile terminal, or to a user's wearable device, or to a server. The wearable device may be a bracelet or a smart watch, etc., and the mobile terminal may be a smartphone used by the user. That is to say, the execution subject of the method may be a mobile terminal, a wearable device, an AV playback device (audio and video playback device), or a server, where the server can obtain data from the mobile terminal and the wearable device. If the method is performed by a mobile terminal, the mobile terminal can establish a connection with the wearable device directly, and obtain data from the wearable device through the established connection. If the method is performed by a wearable device, it is also possible to obtain data from the mobile terminal through an established connection. Of course, a mobile terminal and wearable device can also obtain data from a server—that is, one of the two devices transmits data to the server, and the server forwards the data to the other device. Specifically, the execution subject of the method of the embodiment of the present application may be an electronic device, which may be at least one of the aforementioned mobile terminal, wearable device and server. The method can include the following operations: S301 to S303.

Operation S301 involves acquiring the screen state of a target terminal. This process is described in more detail below.

The target terminal may be at least one of the above-mentioned devices, including a mobile terminal, wearable device, and AV playback device. The screen state may comprise a light-on/light-off state of the screen, an unlocked/locked state of the screen, a touch state, a display state and so on.

The light-on/light-off state of the screen may include a screen-light-on state and a screen-light-off state. The screen displays a bright screen in the screen-light-on state, i.e., the backlight panel of the screen is illuminated, and the screen displays a dark screen in the screen-light-off state, i.e., the backlight panel of the screen is not illuminated or the illumination is weak.

In one example, the screen state may be the screen state of the mobile terminal, or the screen state of the wearable device, or the AV playback device within the user's current environment, and as an implementation, the AV playback device may be a screened device. Specifically, the screened device may be a device with a screen installed, wherein the user's current environment may be determined based on the user's location. In one implementation, the location information of the user can be determined by the user's mobile terminal or wearable device, and the environment in which the user is currently located is determined based on the location information of the user. The screened device within the environment in which the user is currently located can then be predetermined. The devices screened within the user's home environment may be determined based on their home address. For example, after obtaining the user's location information and determining that they are at their home address, the screened devices within their current environment may include a TV set, etc. Alternatively, the user may configure a specific set of screened devices for their home address. For example, even though there may be multiple screened devices at the user's home address, they may choose to select only one or a few of them as the corresponding screened devices. Further, it may be that when the user's location information is determined to be located at the user's home address, the user's location information is further determined to be located in a specific room at the home address based on indoor positioning technology, which is noted as a target room, and the screened device set in the target room is selected as the screened device in the user's current environment.

As an example, the target terminal may be a terminal associated with the user, specifically, the identity information corresponding to the user account logged into the target terminal is the identity information of the user. For instance, the ID of the real-name authentication of the user account may be the same as the ID of the user. In some embodiments, when a user has multiple terminal devices, the target terminal can be determined based on their usage data for each device. Specifically, this usage data includes the user's interactions with each terminal device and can be used to identify the most recently used device as the target terminal. The screen state of this target terminal can then be obtained. In other embodiments, if the use location of the target terminal matches the resident address of the user, the target terminal can be associated with the user. The use location can be the location where the terminal is located when it is used, and the resident address may include the location where the user frequently resides such, as an office address or a home address. For example, if the target terminal is above-mentioned AV playback device, and the installation location of the AV playback device is located within the specified range of the target address of the user, then the AV playback device can be associated with the user. The target address can be the user's home address, and the specified range may be set according to actual need. For example, the AV playback device can be a screened device as described above, and the AV playback device can be associated with the user if the AV playback device is a screened device among the user's smart home device(s). Alternatively, the wearable device can be a device associated with the user's mobile terminal, as generally described in the following embodiments.

As an implementation, if the screen state is a screen state of a mobile terminal or an AV playback device, the screen-light-on state and the screen-light-off state of the screen can be detected by a program module within the system of the mobile terminal or the AV playback device. For example, the screen-light-off state and the screen-light-on state can be detected by the isScreenOn function of a PowerManager. In another implementation, a user's wearable device can collect information on the screen-light-on and screen-light-off states of their mobile terminal or AV playback device. Specifically, the wearable device is equipped with a light sensor that can detect changes in the surrounding light intensity. This allows it to determine the screen state of the mobile terminal or AV playback device if the user is using it while wearing the wearable device or if the wearable device is placed near either of these devices. Since the screen of the mobile terminal or the AV playback device emits different light intensity in the screen-light-on state and the screen-light-off state, the screen-light-on state and the screen-light-off state of the screen can be detected through the collection of the light intensity of the screen of the mobile terminal or the AV playback device by the wearable device.

As mentioned above, the screen state can include an unlocked/locked state of a mobile terminal. In some embodiments, the unlocked/locked state of a screen can be determined based upon the broadcast of an instruction, such as Intent.ACTION_SCREEN_ON and Intent.ACTION_SCREEN_OFF, received by the BroadcastReceiver of the mobile terminal.

The screen state can also include a touch state of a screen, including a touched state and an untouched state. The touched state can refer to the screen being touched within a specified time period, and the untouched state can refer to the screen being touched within a specified time period. For example, a device can detect whether its screen is currently being touched. If it is, the touch state of the screen would be determined to be in a touched state. Otherwise, the touch state would be considered to be in an untouched state.

In another implementation, a device could detect whether its screen has received a touch operation within a specified time period before the current moment. The touch state of the screen would then be determined based on whether any touch operations were detected within this specified time period. In some embodiments, if the screen has received a touch operation within the specified time period (that is, the screen has been touched), the touch state of the screen may be regarded as being in the touched state. If the screen has not received a touch operation within the specified time period, the touch state of the screen may be regarded as the untouched state. In other embodiments, a device could determine the touch state of its screen based on the number of touch operations it has detected within a specified time period. If this number is greater than a threshold, the touch state would be considered to be in a touched state. Otherwise, if the number is less than or equal to this threshold, the touch state would be considered to be in an untouched state.

The display state of the screen can also include a desktop interface state and a non-desktop interface state. A desktop interface state can indicate that the content currently displayed on the screen is a system desktop, displaying icons of applications installed by the target terminal as an operating interface for the user to select and operate the applications from a plurality of applications. A non-desktop interface state is used to indicate that the content currently displayed on the screen is non-system desktop. The non-desktop interface state can also determine a plurality of sub-states according to the type of content displayed, for example, a video interface sub-state, a game interface sub-state, etc. The video interface sub-state can be used to indicate that the interface currently displayed on the screen is a video interface, i.e., the content displayed on the screen is a video, and the game interface sub-state is used to indicate that the interface currently displayed on the screen is a game interface, i.e., the content displayed on the screen is the interface of a game application.

Operation S302 involves obtaining physiological parameters of the user. This process is described in more detail below.

The physiological parameters can include at least one of body motion parameters and a heart rate parameter. Physiological parameters of the user can be obtained through a wearable device of the user. For example, the wearable device can have sensors for acquiring body motion parameters and heart rate parameters of a user. An acceleration sensor can be used to acquire body motion parameters, and a heart rate sensor may be used to acquire heart rate parameters.

In some embodiments, the body motion parameters may be data for characterizing body movement of a user. For example, the body motion parameters may be acceleration data of the user's body, which may be acceleration generated during a physical movement of the user. The acceleration data can include the acceleration values generated during the user's hand movements and the time point corresponding to each acceleration value.

Operation S303 is determining the sleep state of the user based on the screen state and the physiological parameters. This process is described in more detail below.

The sleep state includes a sleeping state and an awake state. As an embodiment, the physiological parameters are capable of determining the sleep state of the user. For example, when the user is asleep, the body movement is small and the heart rate exhibits a resting heart rate. If the physiological parameters include body motion parameters, and the body motion parameters is acceleration data, the user may be considered to be asleep if the acceleration value is quite low, and if the physiological parameters include heart rate parameters, the user may be considered to be asleep if the heart rate parameters match the user's resting heart rate.

As a result, it is possible to determine whether the user is in either a sleeping state or an awake state based on the user's physiological parameters, and then, the screen state can reflect the user's intention to operate the target terminal. Specifically, the operating intention can include an “operating” intention and a “no operating” intention. In particular, if the screen is in a light-on, unlocked or touched state, it can be determined that the user has an intention to operate the device. For example, the screen state can include the light-on/light-off state of the screen, and if the screen state is a screen-light-on state, it can be determined that the user intends to operate the device. Conversely, if the screen state is a screen-light-off state, it can be determined that the user does not intend to operate the device. In another example, the screen state may include an unlocked/locked state of the screen. If the screen is in an unlocked state, it can be determined that the user has the intention to operate the device. On the other hand, if the screen is in a locked state, it can be determined that the user does not have the intention to operate the device. Similarly, if the screen is in the touched state, it can be determined that the user has the intention to operate the device, and if the screen is in an untouched state, it can be determined that the user does not have the intention to operate the device. If there is intention to operate the device, the user can be considered to be in the “awake” sleep state, and if there is no intention to operate the device, the user can be to be in a “sleeping” sleep state.

Therefore, the determination result of the user's sleep state can also be obtained according to the screen state, which is noted as a first result, and the determination result of the user's sleep state will be obtained according to the user's physiological parameters, which is noted as a second result. The sleep state of the user can be determined by combining the first result and the second result.

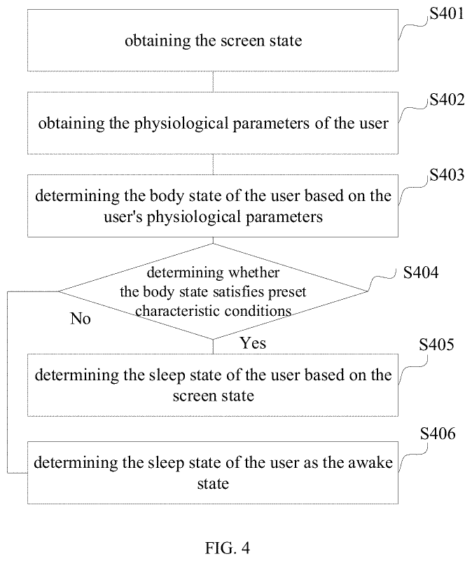

Since the screen state can reflect user's operation of the target terminal, and the operation can further reflect the user's sleep state, the physiological parameters can also reflect the user's sleep state. The combination of the screen state and the physiological parameters can therefore improve the accuracy of a sleep state determination. As shown in FIG. 4, an embodiment of this application provides a method for sleep monitoring. The execution this method can follow the method described in FIG. 3, and therefore will not be repeated here for brevity. Specifically, the method may include operations S401 to S406.

Operation S401 involves obtaining the screen state.

Operation S402 obtaining the physiological parameters of the user.

Operation S403 involves determining the body state of the user based on the user's physiological parameters.

The body state may include a movement state and a heartbeat state. The movement state can correspond to the body motion parameter and the heartbeat state can correspond to the heart rate parameter.

Specifically, the movement state of the user can be determined based on the body motion parameter of the user, which movement state includes a stationary state and a non-stationary state. When the user is in a stationary state, the user's body has a relatively small amplitude of action, indicating that the user is relatively stationary. When the user is in a non-stationary state, the user's body may have a relatively large amplitude of action.

As an implementation, the body motion parameter can be based on acceleration data of a user's limb. The acceleration data of the user may be obtained during a detection period, and if there is a limited change to the acceleration data, and the acceleration value is lower than a specified acceleration value, then the user may be determined to be in the stationary state. Otherwise, the movement state of the user can be determined to be in a non-stationary state. The specified acceleration threshold can be set based on factors such as experience or usage requirements.

In some embodiments, the acceleration data of the user may be collected by a wearable device of the user worn on a limb of the user. For example, the wearable device may be a bracelet or smartwatch, it may be in a sleeping state when the watch or bracelet is worn with a small acceleration value for a sustained (e.g. relatively long) period of time. Specifically, the wearable device may have a three-axis acceleration sensor, providing an acceleration value of each axis for use as acceleration data. The specified acceleration value can include a second threshold value corresponding to each axis, and a determination can be made whether the acceleration value of each axis is less than the specified acceleration value corresponding to that axis. If the acceleration value of each axis is less than the specified acceleration value corresponding to that axis, and lasts for a certain period of time (e.g., 1 minute), then it can be determined that the user's body is in a stationary state. Otherwise, it can be determined that the user's body is in a non-stationary state.

As an implementation, the heartbeat state of the user may comprise a resting state and a non-resting state. After obtaining the heart rate parameter, real-time heart rate information can be determined based on the heart rate parameter, and used to characterize the speed of the user's heartbeat in real time. It should be noted that the real-time heart rate information does not indicate the current heart rate, but may be the heart rate for a period of time up to the current moment. The heart rate parameter can be compared with the resting heart rate, and if there is no significant increase in the heart rate parameter compared to the resting heart rate, then it may be determined that the heartbeat state of the user is a resting state. Otherwise, the heartbeat state of the user can be determined to be in a non-resting state.

As an implementation, the resting heart rate can be the heart rate parameter when the user is in the sleep state during a preset time period, and the resting heart rate can be obtained based on the heart rate parameter when the user is in the sleeping state. The preset time period may be 1 to 3 days before the current moment. A historical resting heart rate can be based upon the heart rate parameter for the user in the sleeping state during the preset time period. To determine if a user's heart rate is in a resting state, the current heart rate can be compared to the historical resting heart rate. A threshold range can be set to define what is considered a significant elevation. The absolute value of the difference between the current heart rate and the historical resting heart rate can be calculated. If this value is less than the resting heartbeat threshold and continues to be so for longer than the resting time threshold, then it can be determined that the user's heartbeat state is in a resting state. Otherwise, it can be determined to be in a non-resting state. As a specific example, the resting heartbeat threshold can be 20 bpm.

Alternatively, in some embodiments, obtaining the physiological parameters of the user is implemented in such a way that the physiological parameters of the user are obtained via a wearable device of the user, and the wearable device of the user having an association with the target terminal. As an implementation, the target terminal may be a mobile terminal, and the mobile terminal can have an association with a wearable device. Specifically, the wearable device can be bound with the mobile terminal while the user is using the wearable device, i.e., the wearable device is considered to have an association with the mobile terminal if the binding relationship exists between the user device and the wearable device. As another implementation, it is also possible that both the mobile terminal and the wearable device require a login user name to be used, and if the current login user name of the mobile terminal is the same as the current login user name of the wearable device, then the wearable device can be considered to be associated with the mobile terminal.

As an implementation, when the wearable device and the mobile terminal are used for monitoring the sleep state of the user, the two need to establish a communication connection, for example, the two are paired via Bluetooth. The worn state of a wearable device can then be determined by checking if it has successfully established a connection with a mobile terminal., i.e., if successful paired, and the wearable device is in the worn state, then operation S401 and subsequent operations can be performed.

Operation S404 involves determining whether the body state satisfies preset characteristic conditions. The preset characteristic conditions may be a body state feature of the user in the sleeping state.

As an example, the body state can be a movement state, and determining whether the body state satisfies the preset characteristic conditions may be to determine whether the body state is in a stationary state. If it is in a stationary state, the body state is determined to satisfy the preset characteristic conditions. Otherwise, the body state is determined to not satisfy the preset characteristic conditions.

As another example, the body state can be a heartbeat state, and the determining whether said body state satisfies the preset characteristic conditions may be to determine whether the heartbeat state is in a resting state. If it is in a resting state, the body state is determined to satisfy the preset characteristic conditions. Otherwise, the body state is determined to not satisfy the preset characteristic conditions.

As another example, the body state can be a movement state and a heartbeat state. Determining whether the body state satisfies the preset characteristic conditions may be to determine whether the body state is in a stationary state and the heartbeat state is in a resting state. If the body state is in a stationary state and the heartbeat state is in a resting state, the body state is determined to satisfy the preset characteristic conditions. Otherwise, the body state is determined to not satisfy the preset characteristic conditions.

Operation S405 involves determining the sleep state of the user based on the screen state. As the specific implementation of determining a screen-light-on state and screen-light-off state can correspond to the aforementioned embodiments, the details will not be repeated here. The user's sleeping state can be determined based on whether the screen is on or off (e.g. based on the aforementioned determination of the light-on/light-off state of the screen).

As an embodiment, the current sleep state of the user may be considered suspected to be a sleeping state if the body state satisfies the preset characteristic conditions. This suspicion can be further supported by the light-on/light-off state of the screen of the target terminal.

In some embodiments, if the screen state is screen-light-off state, then it can be determined that the sleep state of the user is a sleeping state. Since it can be considered that the user is not using the target terminal if the device is in a screen-light-off state, the user can be considered to be asleep if the combined the body state of the user satisfies the preset characteristic conditions.

The light-on/light-off state of the screen of the target terminal can reflect a time period when the user may be asleep. Specifically, the user's awake or sleeping state can be determined based on the screen being on or off for a certain period of time. For example, if the screen is continuously on for more than a set period (e.g., 1 min), it can be considered that the user is awake. If the screen is continuously off for more than a set period, it can be considered that the user is sleeping. A sleep time period Tp can then be determined and sleep time is a subset of Tp. This method can be used to distinguish between scenarios where the user is resting in bed for a long time while swiping through videos on their phone.

As an implementation, the user's sleep state can be determined based on a combination of screen state and physiological parameters, such as body motion and heart rate. The sleep period is determined using these parameters and during this period, the user's sleep state is considered to be sleeping. Outside of the sleep period, the user's sleep state is considered to be awake.

In one example, a first sleep-in time period can be determined based on physiological parameters. A second sleep-in time period can be determined based on the screen state. A target sleep-in time period can be determined based on the first sleep-in time period and the second sleep-in time period. The user's sleep state during the target sleep-in time period can be determined to be the sleeping state. Specifically, a time interval common to the first sleep-in time period and the second sleep-in time period can be used as the target sleep-in time period.

The physiological parameters can be used to determine if a user is in a sleeping state. The physiological parameters collected at each moment can be used to determine the body state. If the body state meets preset characteristic conditions, it can be determined that the user is in a sleeping state. A sleep-in time period can then be determined based on these physiological parameters and recorded as the first sleep time period. As an embodiment, if the physiological parameters include body movement parameters and heart rate parameters, then a third sleep-in time period is determined based on the body movement parameters. A fourth sleep-in time period can be determined based on the heart rate parameters. The time interval common to the third sleep-in time period and the fourth sleep-in time period can be taken as the first sleep-in time period.

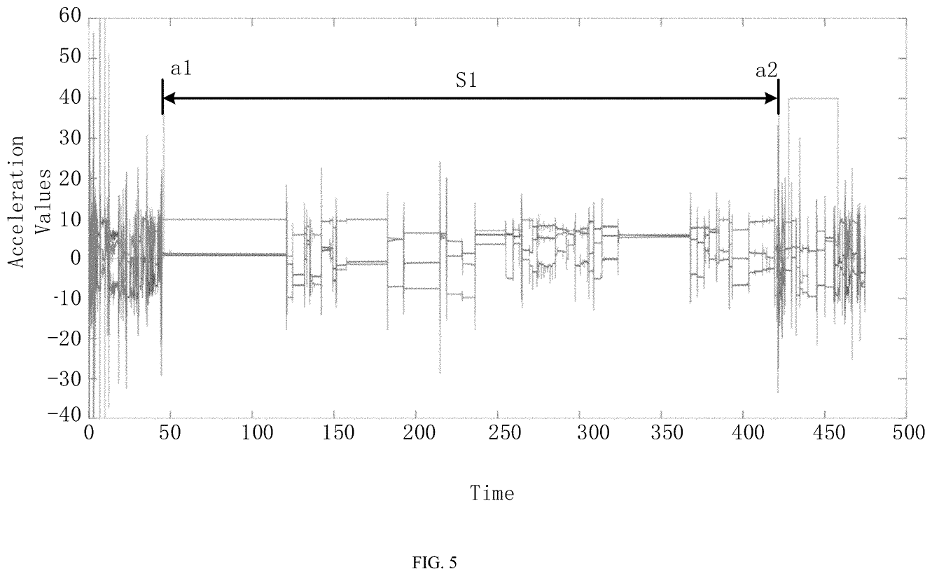

FIG. 5 illustrates a sleep time period S1 of the user determined by acceleration values, and the sleep time period S1 is the third sleep-in time period mentioned above. It can be seen that within S1, the values of acceleration values are relatively small and stable, where a1 is a sleep-in point of the sleep time period S1 and a2 is a wake-up point of the sleep time period S1, i.e., a1 is a first sleep-in point and a2 is a first wake-up point.

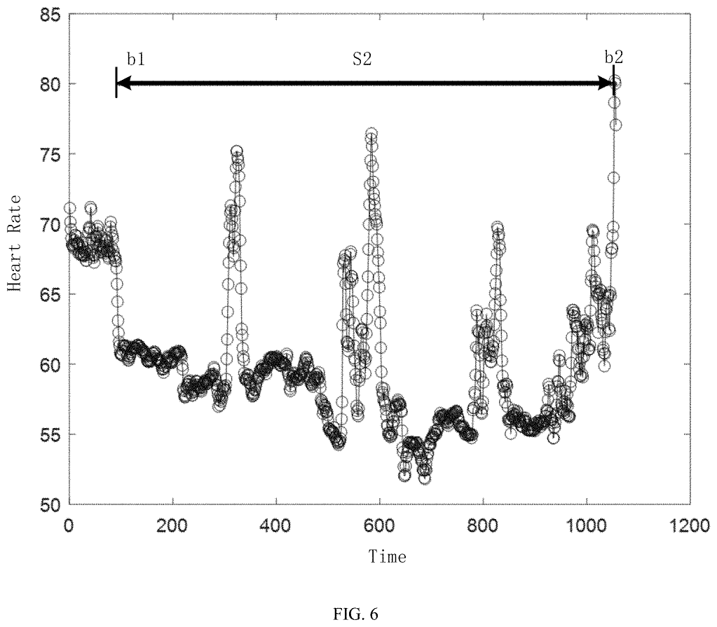

FIG. 6 illustrates a sleep time period S2 of the user determined by heart rate parameter, and the sleep time period S2 is the fourth sleep-in time period mentioned above. It can be seen that within S2, the heart rate is relatively small and closer to the resting heart rate of the user, wherein b1 is a sleep-in point of the sleep time period S2 and b2 is a wake-up point of the sleep time period S2, i.e. b1 is a second sleep-in point and b2 is a second wake-up point.

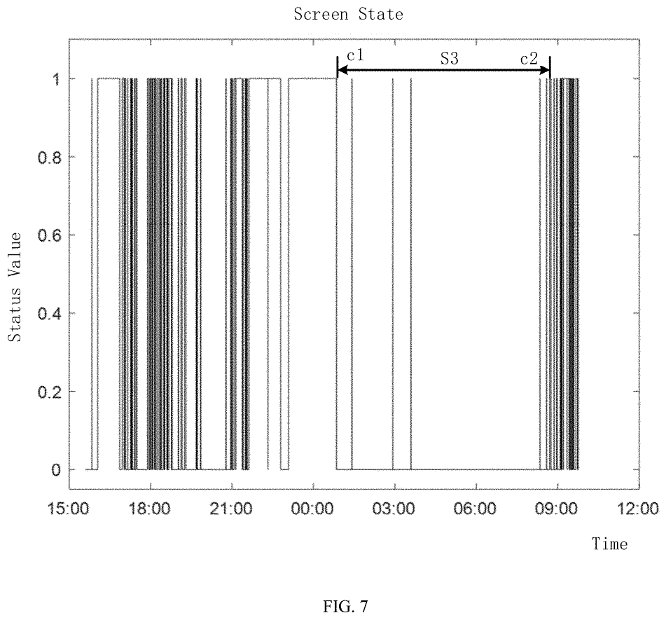

FIG. 7 illustrates a sleep time period S3 of the user determined based on screen state, and the sleep time period S3 is the second sleep-in time period mentioned above. The state value is 1 when the screen is in a screen-light-on state and 0 when the screen is in a screen-light-off state. It can be seen that within S3, the screen is continuously in the screen-light-off state. Further, the status value briefly changes to 1 within S3, as the screen is briefly lit (e.g. when the target terminal receives an alert message or a push message). In the sleep time period S3, c1 represents the point when the user falls asleep and c2 represents the point when the user wakes up. In other words, c1 is the third sleep-in point and c2 is the third wake-up point.

As an implementation, the common time interval between the three sleep-in time periods can be determined by finding their intersection. This common part is the target sleep-in time period and represents the sleep-in time period that satisfies all three methods simultaneously. Specifically, the latest sleep-in point among the first, second and third sleep-in points can be considered as the user's “real” fall asleep point. The earliest wake-up point among the first, second and third wake-up points can be considered as the user's “real” wake-up point. The time period between these two points is the target sleep-in time period.

In some embodiments, if the body state does not satisfy the preset characteristic conditions, and the screen state is determined to be a screen-light-on state, then the sleep state of the user is determined to be an awake state.

In other embodiments, if the screen is on and the user's body state meets preset characteristic conditions, the user's sleep state can be further determined by combining other information. As an implementation, if the user's body state meets preset characteristic conditions and the screen is on, the unlocked/locked state of the screen can be obtained. If the screen is locked, it can be determined that the user is in a sleeping state. If the screen is unlocked, it can be determined that the user is in an awake state.

As another implementation, when the user's body state meets preset characteristic conditions and the screen is on, the display content on the screen can be obtained. If the category of the display content is a given category, it can be determined that the user is in a sleeping state. Otherwise, it can be determined that the user is in an awake state. The given category may be a pre-obtained category of content that easily makes the user fall asleep (e.g. determined by a user, who indicates drama movies as the category) or determined based on statistics from a specified reference time period when the user's body state meets preset characteristic conditions and the screen is on. Thus, multiple content categories for a specified reference time period can be obtained, and the given category can be selected from multiple content categories, for example, the category with the highest frequency of occurrence among the multiple content categories can be determined as the given category.

In addition, the category of the displayed content may be determined based on the format of the content, or it may be determined based on the application corresponding to the content. For example, if the application corresponding to the content is a video category, the category of the content displayed on the screen while the application is running is determined to be a video category. Since users may fall asleep while watching a video, it is possible that when the video playback is completed and the interface automatically exits to display other interfaces within the video application, the user may already be in a sleeping state. Determining the category of displayed content based on the application playing it can make sleep state detection more accurate.

Determining a category of an application may be either the category set by the developer of the application at the time of development or the category set by the user of the application after the application is installed on an electronic device. For example, if the user installs an application on the electronic device, after the installation is completed and the application is accessed, a dialog box is displayed instructing the user to set the category for the application. Then, the user can set the specific category to which an application belongs according to their needs. For example, a social application can be set as an audio, video or social category.

An electronic device has an application installer, such as the Appstore in the IOS system. There is a list of applications in the application installer where users can download applications and update and open them. The application installer can divide different applications into categories, such as audio, video, or games. Thus, the user already knows the category of the application when he installs it with the application installer.

In addition, considering that some applications can play video as well as audio, the category of the application is set to the video category if the application supports the function of video playback, and the category of the application is set to the audio category if it does not support the function of video playback, but only supports the function of audio playback. In particular, it can be determined if an application supports video playback by checking its function description information for supported playback formats. Additionally, the presence of a video playback module within the program module of the application can also indicate support for video playback. For example, a specific video playback codec algorithm may be present, etc.

Further, if some applications have diverse functions, it may be necessary to determine the category of the application based on the specific operation behavior of the application. For example, if some applications can play video and audio, such as some video playback software, can play pure audio files, but also can play video, the application category can be determined based on the usage records of the application. That is, according to the usage records of the application of a certain time period to determine whether the user prefer to play video or audio with the application.

Specifically, the operation behavior data of all users of the application during the predetermined time period is obtained. All users can refer to all users who have installed the application. The operation behavior data can then be obtained from a server corresponding to the application, that is, the user logs into the application with the user account corresponding to the user when using the application, and the operation behavior data corresponding to the user account will be sent to the server corresponding to the application. The server can store the obtained operation behavior data corresponding to the user account. In some embodiments, the electronic device sends an operation behavior query request for the application to the server corresponding to the application, and the server sends all the operation behavior data of the user within a certain preset time period to the electronic device.

The operation behavior data may include the name and time of the audio files played and the name and time of the video files played. By analyzing the operation behavior data, it is possible to determine the number of audio files and the total time played by the application within a predetermined time period. The number of video files and the total time played by the application can also be obtained, in which case, the category of the application can be determined based on the percentage of the total playback time of the audio and video files for the predetermined period of time. Specifically, the percentage of the total playback time of the audio and video files can be obtained within the predetermined time period, and for the purpose of description, record the percentage of the total playback time of the audio files within the predetermined time period as the audio playback percentage. The category of an application can be determined by comparing the percentage of total playback time for video and audio files within a predetermined time period. If the video playback percentage is greater than the audio playback percentage, the application can be categorized as a video application. Otherwise, it may be categorized as an audio application. For example, if the predetermined time period is 30 days (720 hours), and the total playback time for audio files is 200 hours (27.8%) and for video files is 330 hours (45.8%), then the application would be categorized as a video application since the video playback percentage is greater than the audio playback percentage.

As another embodiment, the sleep state of the user can be determined based on real-time heart rate information in the case of the body state of the user satisfies preset characteristic conditions and the screen state is a screen-light-on state. Specifically, if the screen state is determined to be a screen-light-on state, the real-time heart rate information of the user is obtained based on the heart rate parameter. If the real-time heart rate information is less than a first threshold value, then user can be determined to be in the sleeping state. If the real-time heart rate information is not less than the first threshold, then the user can be determined to be in the awake state. The first threshold may be the resting heart rate as previously described.

Specifically, if the physiological parameter used in determining the body state is a body motion parameter, it is possible to determine whether the user is in an awake state or in a sleeping state when the user is suspected of being asleep based on the body motion parameter and when the screen is in screen-light-on state. The real-time heart rate information of the user can be combined to determine whether the sleep state of the user is the awake state or the sleeping state when the amplitude of movement is relatively small and the screen is screen-light-on state. This combination of body motion parameters, screen state, and real-time heart rate information can make sleep state determination more accurate. If the physiological parameters used to determine the body state include real-time heart rate information, the real-time heart rate information can be obtained again when the screen is light-on, thus avoiding inaccurate detection results when the user's heart rate information is detected to indicate that the user is sleeping and the user turns on the target terminal at that time.

As another implementation, it is considered that when the screen light is on, the user may be operating the target terminal with a small amplitude of movement and resting heart rate. If the screen is on, the user's sleep state can be determined based on their heart rate variability information, as detailed in the subsequent embodiments.

Further, it should be noted that, in the case where the body state of the user meets the preset characteristic conditions and the screen state is a screen-light-off state, in addition to the above-mentioned implementation (which can be used to directly determine the sleep state of the user as sleeping state), it is also possible to determine whether the sleep state of the user is the sleeping state or the awake state in the case where the screen state is the screen-light-off state in combination with the heart rate parameters.

As an implementation, if the screen is off, the user's real-time heart rate information can be obtained using heart rate parameters. If the real-time heart rate information is greater than a third threshold, the user's sleep state is determined to be in the awake state. If the real-time heart rate information is not greater than the third threshold, then it can be determined that the user is in the sleeping state. The third threshold may be the same as the first threshold, both of which may be the resting heart rate as described above. Thus, considering that the user is not asleep even though the user is not looking at the screen when the amplitude of body movement is relatively small and the screen is light-off, it is then possible to further determine whether the user is in the sleeping state by real-time heart rate information.

As an implementation, if the screen state is determined to be the screen-light-off state, the heart rate variability information of the user is obtained based on the heart rate parameters, and the sleep state of the user is determined based on the heart rate variability information, specifically, see subsequent implementations.

Operation S406 involves determining the sleep state of the user as the awake state.

If the body state of the user does not meet the preset characteristic conditions, the user's sleep state is determined to be the awake state. It should be noted that the operations not described in detail in the above method can be referred to the preceding embodiments, and therefore will not be repeated here.

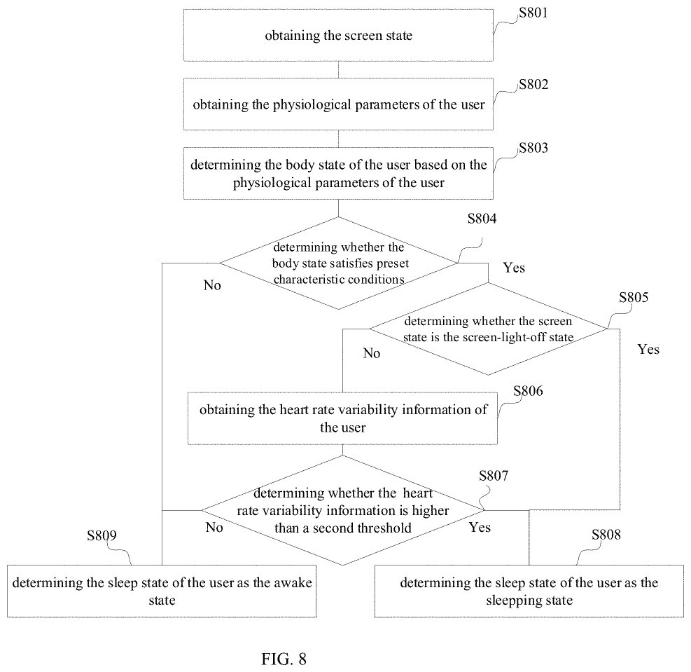

FIG. 8 illustrates a method for sleep monitoring according to an embodiment of the present disclosure. The execution of the method may correspond to the preceding description for the method illustrated in FIG. 3 and FIG. 4, and will therefore not be repeated here. In this application embodiment, the method provides an example where the target terminal is a mobile terminal, and specifically, the method may include: operations S801 to S809.

In operation S801, the screen state is obtained.

In operation S802, the physiological parameters of the user is obtained.

In operation S803, the body state of the user is determined based on the physiological parameters of the user.

In operation S804, whether the body state satisfies preset characteristic conditions is determined.

As an implementation, it may be determined whether the body state of the user satisfies the preset characteristic condition based on either of the acceleration data and the heart rate parameter of the user. The specific implementation of which may correspond to the preceding embodiments.

As an implementation, it can be first determined the current sleep state of the user as sleeping state to be confirmed in the case of that the body state satisfies the preset characteristic condition, i.e., the user is currently in a state of suspected asleep. This can be combined with the screen-light-on/screen-light-off state to more accurately determine a sleep state of the user.

In operation S805, it can be determined whether the screen state is the screen-light-off state.

If the screen state is the screen-light-off state, it can mean that the current user's body state meets the user's body state when sleeping, and, the screen is in the screen-light-off state. Since the possibility of the user using the mobile terminal in the screen-light-off state is very small, it can be considered that the user currently has a great possibility of falling asleep, i.e., operation S808 can be executed.

In addition, considering that when the user is lying in bed, he may just listen to music but not fall asleep. However, at this time the body movement can be relatively small and the heart rate stable. Since a music player can run in the background of a mobile terminal, it is still possible for the mobile terminal to turn off the screen when running the music player application, so at this time, if according to operation S805, an erroneous result would be obtained, i.e. that the the user is in the sleeping state.

In view of the above, it is possible to determine whether the mobile terminal currently has an audio playback-type application running when the judgment result of operation S805 is the screen-light-off state. Specifically, it can be determined whether there is an audio playback-type application running in the background. If there is not, then operation S808 is executed, i.e., it is determined that the sleep state of the user is the sleeping state. If there is, the surrounding ambient sound is collected by the mobile terminal or wearable device, and it is determined whether the surrounding ambient sound is of specified types of voice. For example, the specified types of voice may be snoring. If there is the specified types of voice, operation S808 is executed, i.e., determining that the sleep state of the user is the sleeping state, otherwise, operation S809 is executed, i.e., determining that the sleep state of the user is the awake state.

Operation S806 involves obtaining the heart rate variability information of the user.

If the screen state is screen-light-on state, the heart rate variability information of the user is obtained according to the heart rate parameter. The sleep state of the user can be determined based on the heart rate variability information.

In addition, if the screen briefly lights up due to a push message or notification when the mobile terminal is in screen-off state, it may not be necessary to obtain the user's heart rate variability information and determine their sleep state based on it. This is because the screen lighting up due to a push message or notification is only brief. Therefore, if the screen state is a screen-light-on state, the implementation of obtaining the heart rate variability information of the user may be, if the screen state is the screen-light-on state, to obtain a first time period that the screen state continues to be in the screen-light-on state. If the first time period is greater than a first specified threshold, the heart rate variability information of the user is obtained. The first specified threshold may be set based on experience, for example, setting a relatively small value, such as a 10 second threshold for the first specified threshold. In addition, the first specified threshold may also be set based on the set notification alert period of the mobile terminal. Specifically, this first specified threshold may be greater than or equal to this notification alert length, and therefore, if the first time period of the continuously lit screen is less than or equal to the first specified threshold, it can be considered that the current screen is lit temporarily.

Heart rate variability (HRV) information refers to the variability of the heart rate cycle-by-cycle variation or the variability of the heart rate fast or slow. Autonomic nerves in the human body regulate functions in the body that are not under subjective control, including heart rate, respiration, blood pressure and digestion. There are two types of autonomic nerves, the sympathetic nerve for “fight or flight” and the parasympathetic nerve for “relaxation or digestion. HRV, can reflect the working condition of your autonomic nerves. If the user is in the “fight or flight” sympathetic dominant mode, the user's HRV will be lower, i.e., if the sympathetic nerves are more active, the HRV will be lower, i.e., the more active the sympathetic nerves are, the lower the HRV will be. If the user is in the parasympathetic dominant mode of “relaxation or digestion”, your HRV will be higher. Therefore, HRV can reflect the sympathetic activity of the user.

When the user is in the sleeping state, the sympathetic nerve activity may not be high and the value of HRV can be higher. When the user is in the awake state, the sympathetic nerve activity can be higher even though the body movements are small or the heartbeat is in the resting heartbeat state, and therefore the value of HRV would be lower.

Operation S807 involves determining whether the heart rate variability information is higher than a second threshold.

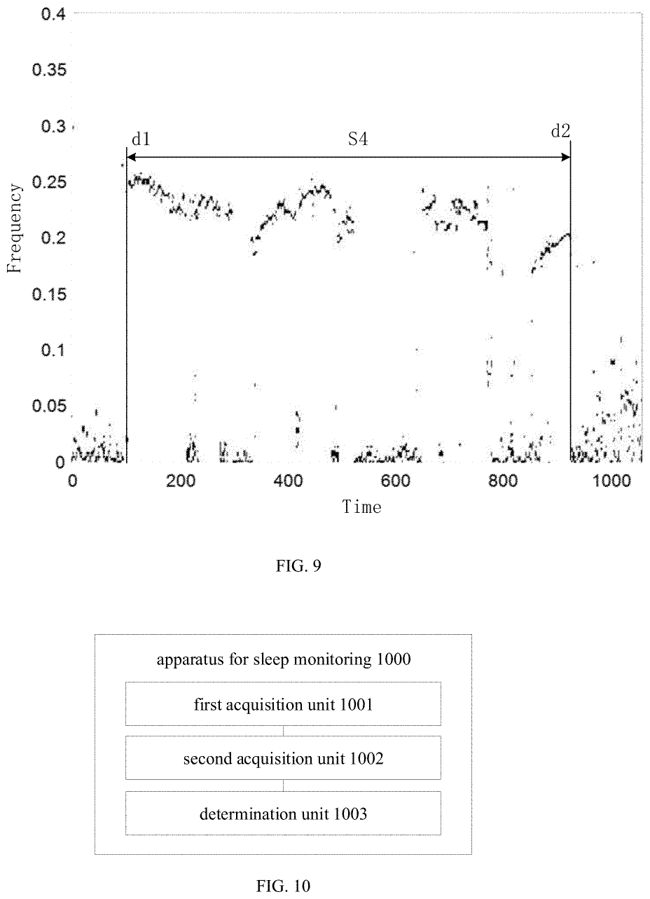

The second threshold can be set according to actual needs, for example, it can be a value between 0.2 kHz and 0.3 kHz. As illustrated in FIG. 9, at the position of d1, the value of HRV increases and shows a high frequency state. It can then be determined that the user is currently in a parasympathetic dominant mode, i.e. in a more relaxed state, and therefore, it can be considered that the user's current sleep state is the sleeping state. At the position of d2, the value of HRV becomes smaller and shows a low frequency state, and it can be determined that the user is currently in a sympathetic dominant mode with sympathetic nerve activity is relatively high and the current sleep state of the user is the awake state.

In S808, the user is determined to be in the sleeping state.

In addition, in the case that the body state meets the predetermined characteristic conditions and the screen state is the screen-light-off state, the heart rate variability information of the user is obtained according to the heart rate parameter. If the heart rate variability information is less than the fourth threshold, it can be determined that the sleep state of the user is the awake state. If the heart rate variability information is not less than the fourth threshold, it can be determined that the sleep state of the user is the sleeping state. The fourth threshold may be the same as the second threshold as previously described, specifically, reference may be made to the aforementioned S806 to S807 and will therefore not be repeated here.

In S809, the user is determined to be in the awake state.

As an implementation, if the screen is on when the user's physiological parameters meet preset characteristic conditions, their heart rate variability information can be obtained and used to determine their sleep state. If the determination result is that the user is asleep, to avoid misjudgment due to a transient elevation of HRV, it can be determined if HRV has been elevated for a certain time period before determining that the user is asleep.

Specifically, a second time period for which the heart rate variability information continues above a second threshold is obtained. If the second time period is greater than a second specified threshold, it can be determined that the sleep state of the user is the sleeping state. The second specified threshold may be set based on experience, for example, it may be from 10 to 20 minutes.

Further, the aforementioned physiological parameters may include at least one of the data of body motion parameters, heart rate parameters, respiration, blood oxygen, body temperature, etc., and may also include the operation data of the wearable device. The operation data of the wearable device may include the screen state of the wearable device, naming the screen state of the mobile terminal as the first screen state and naming the screen state of the wearable device as the second screen state.

Thus, the above-described implementation of determining the sleep state of the user based on the screen state and physiological parameters may be changed to determine the sleep state of the user based on the first screen state, the second screen state and physiological parameters, then the above-described implementation of determining whether the screen state is an screen-light-off state or a screen-light-on state may be changed to determine the light-on/light-off state of the first screen state and the second screen state. The above implementation of determining the sleep state of the user to be the sleeping state if the screen state is screen-light-off state can be changed to determine the sleep state of the user to be the sleeping state if both the first screen state and the second screen state are the screen-light-off state. The above implementation of obtaining the user's heart rate variability information if the screen state is a screen-light-on state is changed to obtaining the user's heart rate variability information if both the first screen state and the second screen state are screen-light-on states, the specific implementation can be replaced with reference to the aforementioned implementation, and will therefore not be repeated here.

FIG. 10 illustrates a structural block diagram of an apparatus for sleep monitoring provided by an embodiment of the present application. The apparatus for sleep monitoring 1000 may include: a first acquisition unit 1001, a second acquisition unit 1002, and a determination unit 1003.

The first acquisition unit 1001 is used to obtain screen state of a target terminal.

The second acquisition unit 1002 is used to obtain physiological parameters of a user, the physiological parameters include at least one of body motion parameters and heart rate parameters.

The second acquisition unit 1002 can be further used to obtain physiological parameters of the user through the wearable device of the user, the wearable device of the user having an association with the target terminal.

Determination unit 1003 is used to determine the sleep state of the user based on the screen state and physiological parameters.

The determination unit 1003 is further used: to determine the body state of the user based on the user's physiological parameters; and to determine whether the body state satisfies a preset characteristic conditions, the preset characteristic conditions being the body state of the user in a sleeping state. If the preset characteristic conditions are satisfied, it can be determined whether the screen state is the screen-light-off state or screen-light-on state. Whether the user is in the sleeping state can be determined based on the result of the determination for the light-on/light-off state of the screen. If the preset characteristic conditions are not satisfied, it can be determined that the user is in the awake state.

The determination unit 1003 is further used to determine that the sleep state of the user is the sleeping state if the screen state is the screen-light-off state.

The determination unit 1003 can be further used to obtain the heart rate variability information of the user if the screen state is a screen-light-on state, the heart rate variability information is used to characterize the sympathetic nerve activity of the user. It can also determine that the sleep state of the user is the sleeping state if the heart rate variability information is above a second threshold; and determine that the sleep state of the user is the awake state if the heart rate variability information is not above the second threshold.

The determination unit 1003 can be further used to obtain a first time period that the screen state has been in the screen-light-on state if the screen state is the screen-light-on state; and to obtain the heart rate variability information of the user if the first time period is greater than a first specified threshold.

The determination unit 1003 is further used to obtain a second time period that the heart rate variability information remains above a second threshold if the heart rate variability information is above a second threshold; and to determine that the sleep state of the user is the sleeping state if the second time period is greater than a second specified threshold.

It will be clear to those skilled in the field that, for the convenience and brevity of the description, the specific working process of the above described apparatus and module can be referred to the corresponding process in the previous method embodiment and will not be repeated herein.

In the embodiments provided by the present application, the modules are coupled to each other either electrically, mechanically or in other forms.

Alternatively, each functional module in each embodiment of the present application may be integrated in a single processing module, or each module may physically exist separately, or two or more modules may be integrated in a single module. The above integrated modules can be implemented either in the form of hardware or in the form of software functional modules.

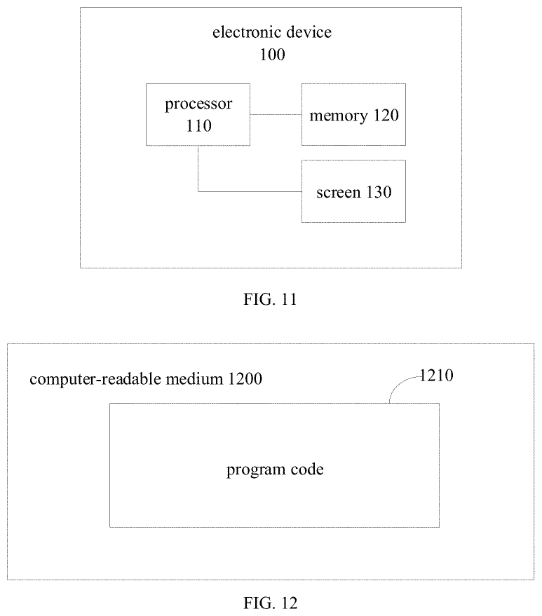

FIG. 11 illustrates a block diagram of the structure of an electronic device provided by an embodiment of the present application. The electronic device 100 may be a target terminal or server as described above.

The electronic device 100 of the present application may include one or more of the following components: a processor 110, a memory 120, a screen 130, and one or more applications. The one or more applications may be stored in the memory 120 and configured to be executed by the one or more processors 110. The one or more applications can be configured to perform the method as described in the precede ng embodiments of method.

The processor 110 may include one or more processing cores. The processors 110 utilize various interfaces and lines to connect various parts within the entire electronic device 100 to perform various functions and process data of the electronic device 100 by running or executing instructions, programs, code sets, or instruction sets stored in the memory 120, and by calling data stored in the memory 120. In at least one embodiment, the processor 110 may employ at least one of Digital Signal Processing (DSP), Field-Programmable Gate Array (FPGA), Programmable Logic Array (PLA) to implement at least one of the hardware forms. The processor 110 may integrate one or a combination of one or more of a Central Processing Unit (CPU), a Graphics Processing Unit (GPU), and a modem, etc. Among these, the CPU primarily handles the operating system, user interface, applications, etc.; the GPU is used to take care of rendering and drawing of display content; and the modem is used to handle wireless communication. It can be appreciated that the above modem can also be implemented without being integrated into the processor 110 and through a separate communication chip.

The memory 120 may include Random Access Memory (RAM), or it may include Read-Only Memory (ROM). Memory 120 may be used to store instructions, programs, code, code sets, or instruction sets. Memory 120 may include a program storage area and a data storage area, wherein the program storage area may store instructions for implementing an operating system, instructions for implementing at least one function (e.g., a touch function, a sound playback function, an image play function, etc.), instructions for implementing each of the method embodiments described below, etc. The data storage area may also store data created by the electronic device 100 in use (e.g., phone book, audio and video data, chat log data), etc.

Referring to FIG. 12, which illustrates a block diagram of the structure of a computer-readable medium provided by an embodiment of the present application. The computer-readable medium 1200 has program code stored in it, and the program code can be called by a processor to execute the method described in the embodiment of method above.

The computer-readable medium 1200 may be an electronic memory such as a flash memory, an EEPROM (electrically erasable programmable read-only memory), an EPROM, a hard disk, or a ROM. In at least one embodiment, the computer-readable medium 1200 includes a non-transitory computer-readable storage medium. The computer-readable medium 1200 has storage space for program code 1210 that performs any of the method operations of the method described above. The program code may be read from or written to one or more computer program products. The program code 1210 may be compressed, for example, in an appropriate form.

Finally, it should be noted that the above embodiments are intended only to illustrate the technical solutions of the present application and not to limit them; although the present application is described in detail with reference to the foregoing embodiments, it is understood by a person of ordinary skill in the art that it is still possible to modify the technical solutions described in the foregoing embodiments or to replace some of the technical features thereof with equivalent ones; and these modifications or replacements do not drive the nature of the corresponding technical solutions out of the respective embodiments of the present application. These modifications or substitutions do not drive the essence of the technical solutions away from the spirit and scope of the technical solutions of the embodiments of the present application.

What is claimed is:

1. A method for sleep monitoring by an electronic device, comprising:
acquiring a screen state of a target terminal;
obtaining physiological parameters of a user, the physiological parameters including at least one of body motion parameters and heart rate parameters; and
determining a sleep state of the user based on the screen state and the physiological parameters, the sleep state comprising a sleeping state and an awake state.

2. The method of claim 1, wherein determining the sleep state of the user based on the screen state and the physiological parameters further comprises:
determining the body state of the user based on the physiological parameters, the body state comprising at least one of a movement state and a heartbeat state;
determining whether the body state satisfies a preset characteristic condition, the preset characteristic condition being a characteristic of a body state corresponding to the sleep state;
determining the sleep state of the user based on the screen state if the body state satisfies the preset characteristic condition; and
determining the sleep state of the user to be the awake state if the body state does not satisfy the preset characteristic condition.

3. The method of claim 2, wherein determining the sleep state of the user based on the screen state further comprises:
determining the sleep state of the user to be the sleeping state if the screen state is determined to be a screen-light-off state; and
determining the sleep state of the user to be the awake state if the screen state is determined to be a screen-light-on state.

4. The method of claim 2, wherein the physiological parameters comprising heart rate parameters, and determining the sleep state of the user based on the screen state, further comprises:
determining the sleep state of the user to be the sleeping state if the screen state is determined to be a screen-light-off state;
obtaining real-time heart rate information of the user based on the heart rate parameters if the screen state is determined to be a screen-light-on state;
determining the sleep state of the user to be the sleeping state if the real-time heart rate information is less than a first threshold; and
determining the sleep state of the user to be the awake state if the real-time heart rate information is not less than the first threshold.

5. The method of claim 2, wherein the physiological parameters comprising heart rate parameters, and determining the sleep state of the user based on the screen state, further comprises:
determining the sleep state of the user to be the sleeping state if the screen state is determined to be a screen-light-off state;
obtaining heart rate variability information of the user based on the heart rate parameters if the screen state is determined to be a screen-light-on state;
determining the sleep state of the user to be the sleeping state if the heart rate variability information is greater than a second threshold; and
determining the sleep state of the user to be the awake state if the heart rate variability information is not greater than the second threshold.

6. The method of claim 2, wherein the physiological parameters comprising heart rate parameters, and determining the sleep state of the user based on the screen state, further comprises:
determining the sleep state of the user to be the awake state if the screen state is determined to be a screen-light-on state;
obtaining real-time heart rate information of the user based on the heart rate parameters if the screen state is determined to be a screen-light-off state;
determining the sleep state of the user to be the awake state if the real-time heart rate information is greater than a third threshold; and
determining the sleep state of the user to be the sleeping state if the real-time heart rate information is not greater than the third threshold.

7. The method of claim 2, wherein the physiological parameters comprising heart rate parameters, and determining the sleep state of the user based on the screen state, further comprises:
determining the sleep state of the user to be the awake state if the screen state is determined to be a screen-light-on state;
obtaining heart rate variability information of the user based on the heart rate parameters if the screen state is determined to be a screen-light-off state;
determining the sleep state of the user to be the awake state if the heart rate variability information is less than a fourth threshold; and
determining the sleep state of the user to be the sleeping state if the heart rate variability information is not less than the fourth threshold.

8. The method of claim 2, wherein determining the sleep state of the user based on the screen state further comprises:
determining the sleep state of the user to be the sleeping state if the screen state is an untouched state; and
determining the sleep state of the user to be the awake state if the screen state is a touched state.

9. The method of claims 2, wherein determining the sleep state of the user based on the screen state further comprises:
determining the sleep state of the user to be the sleeping state if the screen state is a locked state; and
determining the sleep state of the user to be the awake state if the screen state is an unlocked state.

10. The method of claim 2, wherein the body state comprising the movement state, and determining whether the body state satisfies the preset characteristic condition, further comprises:
determining whether the movement state is a stationary state;
determining that the body state satisfies the preset characteristic condition if the movement state is the stationary state; and
determining that the body state does not satisfy the preset characteristic condition if the movement state is a non-stationary state.

11. The method of claim 2, wherein the body state comprising the heartbeat state, and determining whether the body state satisfies the preset characteristic condition, further comprises:
determining whether the heartbeat state is a resting state;
determining that the body state satisfies the preset characteristic condition if the heartbeat state is the resting state; and
determining that the body state does not satisfy the preset characteristic condition if the heartbeat state is a non-resting state.

12. The method of claim 2, wherein the body state comprising the movement state and the heartbeat state, and determining whether the body state satisfies the preset characteristic condition, further comprises:
determining whether the movement state is a stationary state and the heartbeat state is a resting state;
determining that the body state satisfies the preset characteristic condition if the movement state is the stationary state and the heartbeat state is the resting state; and
otherwise, determining that the body state does not satisfy the preset characteristic condition.

13. The method of claim 2, wherein determining the sleep state of the user based on the screen state and the physiological parameters further comprises:
determining whether the user is currently in a target sleep time period;
determining the sleep state of the user to be the sleeping state if the user is currently in the target sleep time period; and
determining the sleep state of the user to be the awake state if the user is not currently in the target sleep time period.

14. The method of claim 13, wherein before determining whether the user is currently in the target sleep time period, further comprises:
determining a first sleep-in time period based on the physiological parameters, and determining a second sleep-in time period based on the screen state; and
determining the target sleep-in time period based on the first sleep-in time period and the second sleep-in time period.

15. The method of claims 14, wherein determining the first sleep-in time period based on the physiological parameters further comprises:
determining a third sleep-in time period based on the body motion parameters;
determining a fourth sleep-in time period based on the heart rate parameters;
obtaining a time interval common to the third sleep-in time period and the fourth sleep-in time period; and
configuring the time interval as the first sleep-in time period.

16. The method of claim 4, wherein determining that the screen state is the screen-light-on state, further comprises:
determining the screen state to be the screen-light-on state if the screen state is continuously in the screen-light-on state for a period longer than a fifth threshold.

17. The method of claim 1, wherein acquiring the screen state of the target terminal, further comprises:
acquiring the screen state of the target terminal associated with the user, the target terminal comprising at least one of a mobile terminal, a wearable device and an AV playback device;
obtaining physiological parameters of a user, comprises:
obtaining the physiological parameters of the user via a wearable device worn by the user, wherein there is an association between the target terminal and the wearable device.

18. An electronic device for sleep monitoring, comprising:
one or more processors and a memory, the memory being configured to store computer program code of one or more applications, which when executed by the one or more processors causes the electronic device to:
obtain a screen state of a target terminal;
obtain physiological parameters of a user, the physiological parameters comprising at least one of body motion parameters and heart rate parameters; and
determine a sleep state of the user based on the screen state and the physiological parameters, the sleep state comprising a sleeping state and an awake state.

19. The electronic device of claim 18, wherein the memory being configured to store computer program code of one or more applications, which when executed by the one or more processors causes the electronic device to:
determine the body state of the user based on the physiological parameters, the body state comprising at least one of a movement state and a heartbeat state;
determine whether the body state satisfies a preset characteristic condition, the preset characteristic condition being a characteristic of a body state corresponding to the sleep state;
determine the sleep state of the user based on the screen state if the body state satisfies the preset characteristic condition;
determine the sleep state of the user to be the awake state if the body state does not satisfy the preset characteristic condition.

20. A non-transitory computer-readable medium storing a program code, which when executed by a processor, causes the processor to: acquire a screen state of a target terminal;
obtain physiological parameters of a user, the physiological parameters including at least one of body motion parameters and heart rate parameters; and
determine a sleep state of the user based on the screen state and the physiological parameters, the sleep state comprising a sleeping state and an awake state.
